(19) United States
(12) Patent Application Publication
(10) Pub. No.: US 2025/0295437 A1
(43) Pub. Date: Sep. 25, 2025

(54) LOCKING FIXATION DEVICE

(71) Applicant: ATLAS SPINE, INC., Jupiter, FL (US)

(72) Inventor: Matthew G. Baynham, Jupiter, FL (US)

(21) Appl. No.: 18/614,027

(22) Filed: Mar. 22, 2024

(51) Int. Cl. A61B 17/70; A61F 2/44

(52) U.S. Cl. CPC A61B 17/7064; A61F 2/446

(57) ABSTRACT

A locking fixation device formed from a malleable wedge-shaped implant constructed from a continuous piece of material adapted to be applied to the human spine for fusing vertebral bodies together. The device is defined by an insert end spaced apart from a spacer end by first and second side members. Each side support is cut by a series of recesses with uniform thickness forming a sinusoidal pattern extending therefrom and perpendicular thereto. The fixation device includes a first and second receiving hole adapted to receive a first and second bone screw for securing to a vertebral body. A locking member rotatably secured to the spacer end includes tabs constructed and arranged to rotate from an installation position allowing insertion of the bone screws to a locking position to prohibit bone screw movement.

### FIELD OF THE INVENTION

The present invention relates generally to surgically-implantable spinal devices and, more specifically, to a locking fixation device formed from a spinal implant with a malleable body.

### BACKGROUND OF THE INVENTION

The human spine contains several types of joints that contribute to its stability, flexibility, and overall function of the spine. Facet joints (Zygapophyseal Joints) connect adjacent vertebrae. Synovial joints have a membrane that produces synovial fluid, facilitating smooth movement between the articular surfaces of the vertebrae. These facet joints play a crucial role in guiding and limiting the movement of the spine, including flexion, extension, rotation, and lateral bending.

Intervertebral discs are fibrocartilaginous structures located between adjacent vertebrae. The intervertebral discs serve as shock absorbers, providing cushioning and support to the spine during various activities. Intervertebral discs contribute to the flexibility and mobility of the spine. The sacroiliac joint (SI Joint) is located at the junction of the sacrum and ilium in the pelvis. While not directly on the back of the spine, the SI joint plays a significant role in the stability and movement of the lower spine and pelvis. It primarily absorbs shock and transfers forces between the upper body and the legs during weight-bearing activities.

Atlanto-occipital Joint and Atlanto-axial Joint are located between the skull and the first cervical vertebra (C1, the atlas), and between the first and second cervical vertebrae (C1 and C2, the axis), respectively. They contribute to the mobility and stability of the neck and head. These various joints in the back of the spine work together to support the body, facilitate movement, and protect the spinal cord while allowing for flexibility and mobility in different regions of the spine. Dysfunction or injury to these joints can lead to pain, stiffness, and reduced mobility in the spine and surrounding areas.

There are numerous implants and associated methods for performing stabilization and/or immobilization in the spine. Conventional implants utilize bone screws that are threaded through the superior and inferior facets to immobilize the facet joint so as to permit the adjoined bone sections to fuse together.

U.S. Pat. No. 9,629,727 discloses a facet joint fixation device formed from a wedge shaped implant constructed from a single piece of material where each side support is cut by a series of recesses.

What is needed is a malleable fixation device that implements an interlocking component to prevent bone screws from backing out.

### SUMMARY OF THE INVENTION

Embodiments of the invention are directed, inter alia, to a fixation device that consists of a malleable wedge-shaped implant that is anchored by a pair of bone screws. The fixation device is constructed from a single piece of material having an insert end spaced apart from outer end by first and second arcuate side members. Each side support is cut like a sinusoidal or accordion shape therebetween. The sinusoidal shape can be placed in a particular shape, retaining the thickness, to provide a spring type resilience.

Upon insertion, a first and second bone screw is positioned through a spacer end. The accordion shape provides a malleable member that allows a surgeon to implant and gain access from unobstructed angles to conform to the spacing of a human facet joint or disc.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a front view of a locking fixation device;

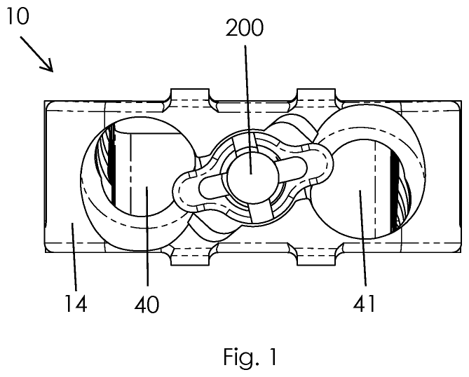

FIG. 2 is a front view thereof;

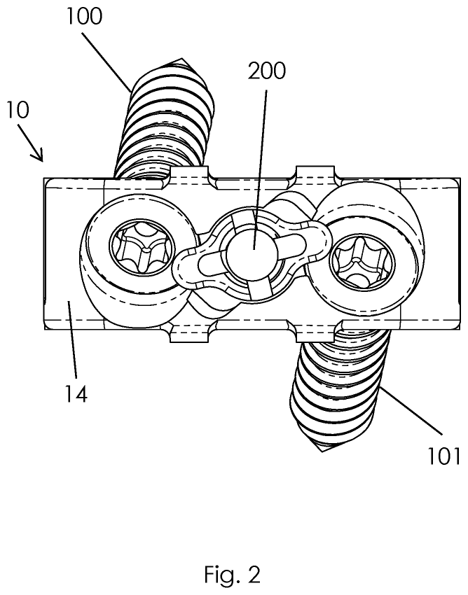

FIG. 3 is a side view thereof;

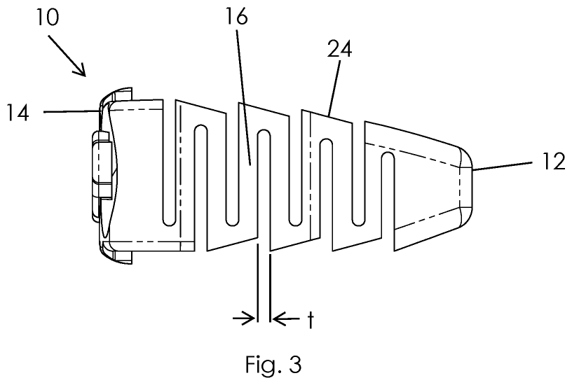

FIG. 4 is a side view thereof;

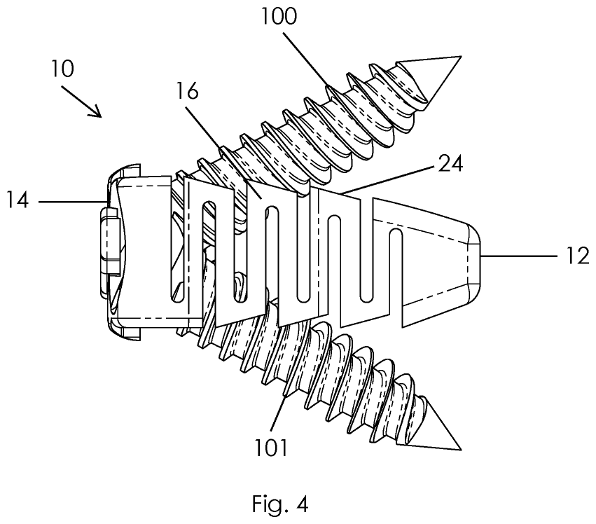

FIG. 5 is a top view thereof;

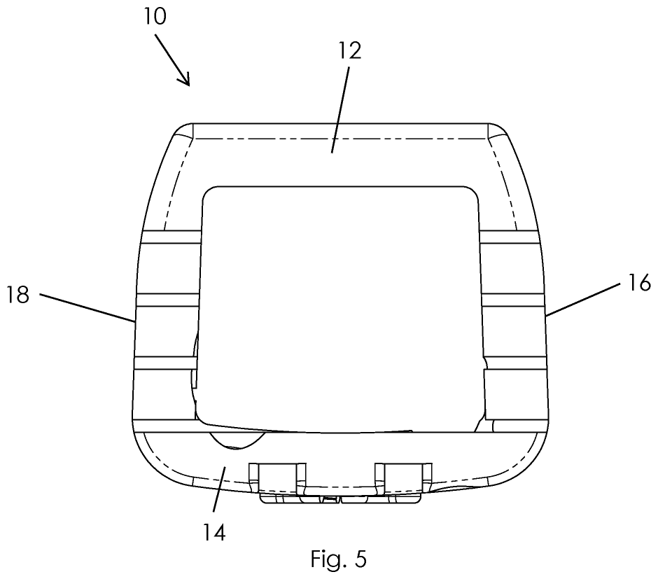

FIG. 6 is a top view thereof with bone screws;

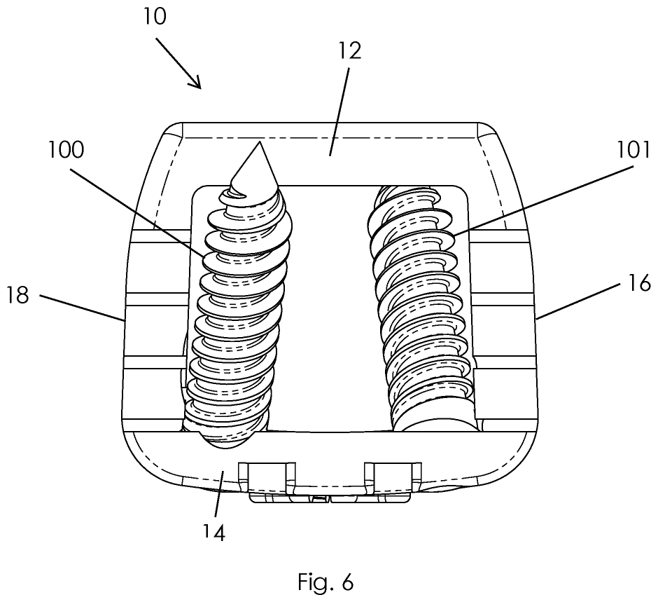

FIG. 7 is a perspective view thereof;

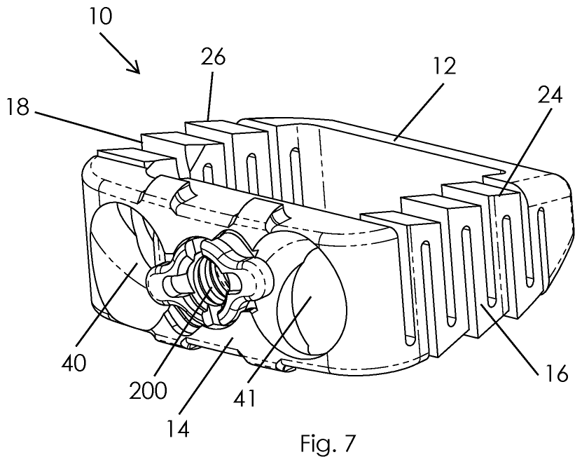

FIG. 8 is a perspective view thereof;

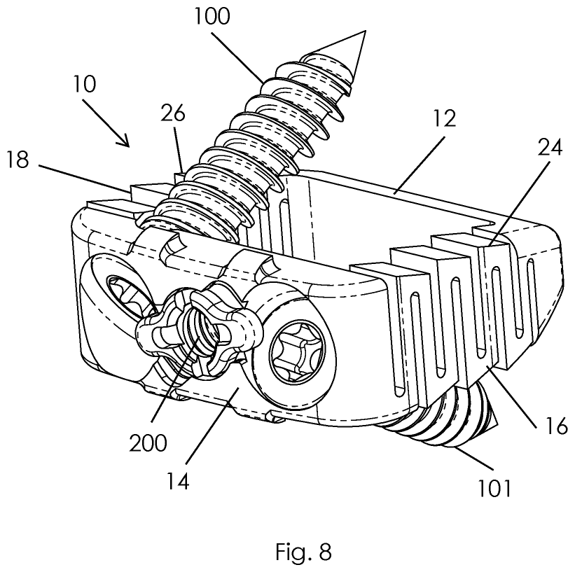

FIG. 9 is an exploded view thereof;

FIG. 10 is a detailed view of the fixation device showing the locking mechanism;

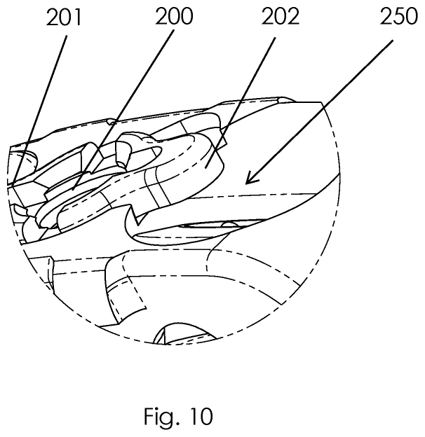

FIG. 11 is a cross-sectional view thereof; and

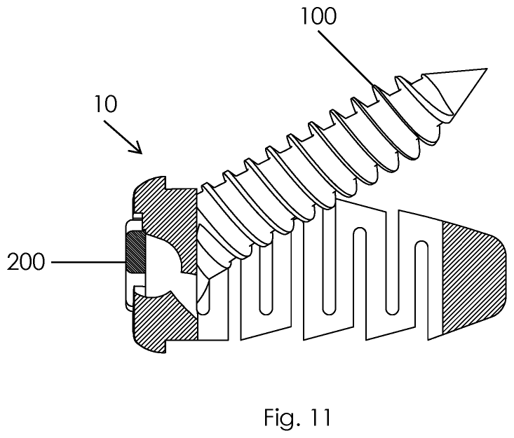

FIG. 12 is a detailed view thereof illustrating a locking mechanism to retain a bone screw from backing out.

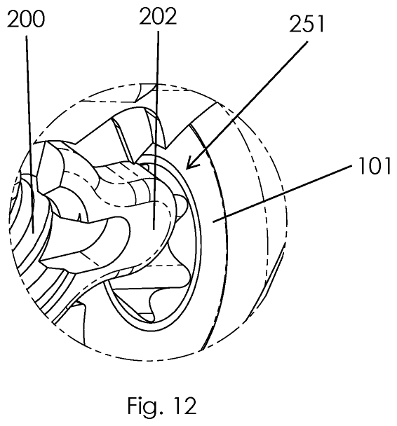

### DETAILED DESCRIPTION OF THE INVENTION

It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The following description of the preferred embodiments is merely exemplary in nature and is in no way intended to limit the invention, its application or uses.

Referring now to the Figures in general, disclosed is a facet joint fixation device that consists of a malleable wedge-shaped implant 10 that can provide anchoring for a bone screw implant. The facet fixation device is preferably constructed from a single piece of biocompatible materials, such as titanium, or any conventional material used for surgical implants, such as stainless steel and its many different alloys, titanium alloys, metallic alloys, polymeric materials, plastics, plastic composites, ceramic and any other metal or material with the requisite strength and biologically inert properties. However, it is to be understood, that the various parts of the device may be constructed from various materials in some embodiments. For example, the side members may be made from a material that provides the requisite strength but also malleability, whereas the insert end and spacer end are made from a rigid material that can be subjected to force without bending or buckling. If desired, the insert end and/or the side members can be coated with a lubricant or material that provides a lubricating effect. In some embodiments, the insert end and/or the spacer end may comprise one or more layers of materials, such as, for example, plastic, polymers, metals or any other biocompatible conventional material(s). In other embodiments, the device may be coated with a biocompatible material, for example, medical grade thermoplastic elastomeric compounds.

The malleable wedge-shaped implant 10 is defined by an insert end 12 spaced apart from an outer or spacer end 14 by a first 16 and second 18 side members forming an accordion like shape having a thickness. Each side member is further defined as a continuous piece of material having a first 16 side surface with a first series of recesses 24 forming a sinusoidal pattern extending therefrom and perpendicular thereto. The recesses 24, 26 can be any shape and size as long as they provide flexibility to the side members. In addition, the recesses 24, 26 serve the purpose of stabilizing or provide grip to prevent slippage or movement of the device. Each side member 16 and 18 is capable of being bent back and forth due to the retained thickness which provide spring hinge type resilience. The first 16 side surface has a slope between adjacent recesses 24, and a second series of recesses 26 extend from a second 18 side surface positioned adjacent to the first series of recesses 24 with the second 18 side surface having a slope between adjacent recesses 26. The thickness of the side member 16, 18 is uniform between the spacer end 14 and the insert end 12.

In preferred embodiments, the side members can flex at least about 20° relative to a horizontal axis, preferably about 45° relative to a horizontal axis, preferably about 50° relative to a horizontal axis, preferably about 75° relative to a horizontal axis, preferably 90° relative to a horizontal axis, preferably about 180° relative to a horizontal axis. In some embodiments, the flexibility is about 270° relative to a horizontal axis.

The fixation device 10 includes a first receiving hole 40 extending through the spacer end 14 from an upper surface 20 through a lower surface 22. The first receiving hole 40 is adapted to receive a first bone screw 100 for securing to a vertebral body. Likewise, the fixation device 10 includes a second receiving hole 41 extending through the spacer end 14 from an upper surface 20 through a lower surface 22. The second receiving hole 41 is adapted to receive a second bone screw 101 for securing to an adjacent vertebral body. In an alternative embodiment, the first receiving hole 40 is formed at a first angle and the second receiving hole 41 is formed at a second angle traverse to the first angle.

To ensure that either bone screw 100, 101 does not reverse direction and remains in place after inserted between two vertebral bodies, a locking member 200 is rotatably secured to the spacer end 14 positioned between the first 40 and second 41 bone screw opening. The locking member 200 includes tabs 201, 202 constructed and arranged to rotate from an installation position 250 allowing insertion of said bone screws to a locking position 251 to prohibit bone screw movement. In a preferred embodiment, the locking member 200 includes a base 204 that has a raised section elevating the locking member 200 into the installation position 250. Rotation of the locking member 200 from the installation position 250 to the locking position 251 bias the locking member 200 in the locking position 251 wherein each the tabs 201, 202 are over the tops of the bone screws 100, 101.

Accordingly, in preferred embodiments, a facet spacer device 10 comprises an insert end 12, a spacer end 14, wherein the insert end 12 and the spacer end 14 are connected by a first 16 and a second 18 side member, wherein each side member 16, 18 comprises at least one recess 24, 26 defining a hinge structure. In a preferred embodiment, the side members 16, 18 comprise a plurality of recesses 24, 26 defining a hinge structure forming a sinusoidal pattern extending therefrom and perpendicular thereto.

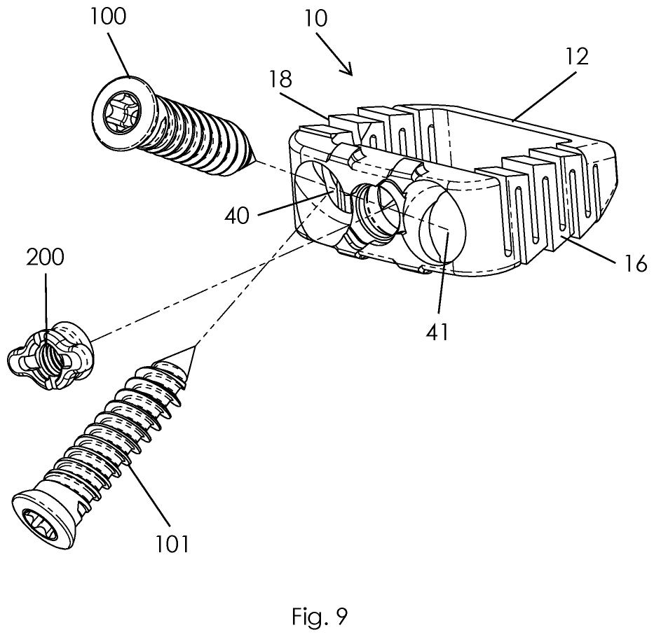

In a preferred embodiment, the insert end 12 comprises a tapered end and the spacer end 14 comprises a curved surface or a planar surface having flat or curved sides or combinations thereof, and at least one receiving hole 40, 41 for receiving a bone screw 100, 101 or other instruments. In a preferred embodiment, the fixation device 10 includes a first receiving hole 40 adapted to receive a first bone screw 100 and a second receiving hole 41 adapted to receive a second bone screw 101 to secure two vertebral bodies, as shown in FIG. 9.

In preferred embodiments, the spacer end 14 is wider than the height of the side members 16, 18. The side members 16, 18 connecting the insert end 12 and the spacer end 14 are attached to the spacer end 14, in a position equidistant from each end of the planar or curved surface. Accordingly, the height of the spacer end 14 is greater than the height of the side members 16, 18 by at least about 0.1 fold to about 10 fold.

Embodiments are also directed to methods and procedures for using the device. In a preferred embodiment, a method of implanting a bone screw in a patient in need thereof, comprises inserting a malleable wedge-shaped implant between adjacent vertebrae, the malleable wedge-shaped implant comprising an insert end, an outer end, wherein the insert end and the outer end are spaced apart by a first and a second side member, each side member having one or more recesses forming an accordion like shape having a thickness, defined as a continuous piece of material having a first side surface with a first series of recesses forming a sinusoidal pattern extending therefrom and perpendicular thereto. The insert end comprises a tapered end for ease of inserting the device in between adjacent vertebrae.

In preferred embodiments, the outer end comprises a planar surface having flat or curved sides or combinations thereof, and at least one receiving hole for receiving the bone screw. It is to be understood that the openings may vary in size for receiving various surgical or other instruments.

In embodiments, the recesses defining the accordion like shaped structure further provide an anchoring thereby preventing the device from moving during the procedure or bone screw pullout.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is to be understood that while a certain form of the invention is illustrated, it is not to be limited to the specific form or arrangement herein described and shown. It will be apparent to those skilled in the art various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A fixation device adapted to be applied to the human spine for fusing vertebral bodies together, said fixation device comprising:
a fixation body having an insert end spaced apart from a spacer end by a side member forming an accordion like shape having a thickness, said side member further defined as a continuous piece of material having a first side surface with a first series of recesses forming a sinusoidal pattern extending therefrom and perpendicular thereto, said first side surface having a slope between adjacent recesses, and a second series of recesses extending from a second side surface positioned adjacent said first series of recesses with said second side surface having a slope between adjacent recesses, said thickness of said side member is uniform between said spacer end and said insert end;
a first receiving hole extending through said spacer end from an upper surface through a lower surface, said first receiving hole being adapted to receive a first bone screw for securing to a vertebral body;
a second receiving hole extending through said spacer end from an upper surface through a lower surface, said second receiving hole being adapted to receive a second bone screw for securing to an adjacent vertebral body;
a locking member rotatably secured to said spacer end positioned between said first and second bone screw opening, said locking member having tabs constructed and arranged to rotate from an installation position allowing insertion of said bone screws and a locking position to prohibit bone screw movement.

2. The fixation device of claim 1, wherein said first receiving hole is formed at a first angle and said second receiving hole is formed at a second angle traverse to said first angle.

3. The fixation device of claim 1 wherein a base of said locking member has a raised section elevating said locking member in said installation position.

4. The fixation device of claim 3 wherein rotation of said locking member from said installation position to said locking position bias said locking member in the locking position wherein each said tab is over the top of a bone screw.

5. The fixation device of claim 1, wherein the insert end comprises a tapered end.

6. The fixation device of claim 1, wherein the spacer end is wider than the height of the side members by at least about 0.1 fold to about 10 fold.

7. The fixation device of claim 1, wherein the insert end and the spacer end are rigid.
